(19) United States
(12) Patent Application Publication
(10) Pub. No.: US 2019/0282215 A1
(43) Pub. Date: Sep. 19, 2019

(54) ULTRASOUND PROBE AND CONTROL METHOD THEREOF

(71) Applicant: Industrial Technology Research Institute, Hsin-Chu (TW)

(72) Inventors: CHUN-JUNG CHEN, Hsinchu County (TW); GUO-ZUA WU, Taichung City (TW)

(21) Appl. No.: 16/225,291

(22) Filed: Dec. 19, 2018

Related U.S. Application Data
(60) Provisional application No. 62/610,969, filed on Dec. 28, 2017.

(30) Foreign Application Priority Data
Dec. 17, 2018 (TW) 107145498

(51) Int. Cl. A61B 8/00; A61B 8/14; A61B 8/06

(52) U.S. Cl. CPC A61B 8/54; A61B 8/06; A61B 8/14

(57) ABSTRACT

An ultrasound probe is provided, which includes a transmitter, a receiver and a controller. The transmitter includes a signal receiving element. The transmitter includes a plurality of signal transmitting elements; there are included angles between each of the signal transmitting elements and the signal receiving element, and the included angles are different from each other. The controller is connected to the transmitter and the receiver; the controller sequentially drives the signal transmitting elements and the signal receiving element receives the echo signal of each signal transmitting elements. The controller makes a comparison among the echo signals and selects one of the signal transmitting elements as a target signal transmitting element according to the comparison result; then, the controller generates a measurement result according to the echo signal of the target signal transmitting element.

### CROSS REFERENCE TO RELATED APPLICATION

The present application is based on, and claims priority from, U.S. Provisional Application No. 62/610,969, filed on Dec. 28, 2017, and Taiwan Application No. 107145498, filed on Dec. 17, 2018, the disclosures of which are hereby incorporated by reference herein in their entirety.

### TECHNICAL FIELD

The technical field relates to an ultrasound probe, in particular to a multi-angle ultrasound probe. The technical field further relates to the control method of the multi-angle ultrasound probe.

### BACKGROUND

The principle of an ultrasound probe measuring distance is to transmit ultrasonic waves to a target object by a signal transmitter element and receive echo signals reflected from the target object by a signal receiver. Then, the distance between the ultrasound probe and the target object can be calculated according to the echo signals. Currently, ultrasound probes have been comprehensively applied to blood flow velocity measurement or other applications.

### SUMMARY

An embodiment of the present disclosure relates to an ultrasound probe, which includes a transmitter, a receiver and a controller. The transmitter includes a signal receiving element. The transmitter includes a plurality of signal transmitting elements. There are included angles between each of the signal transmitting elements and the signal receiving element, and the included angles are different from each other. The controller is connected to the transmitter and the receiver. The controller sequentially drives the signal transmitting elements, and the signal receiving element receives the echo signal of each of the signal transmitting elements. The controller makes a comparison among the echo signals and the controller selects one of the signal transmitting elements as a target signal transmitting element according to the comparison result. Then, the controller generates a measurement result according to the echo signal of the target signal transmitting element.

Another embodiment of the present disclosure relates to a control method for an ultrasound probe, which includes the following steps: sequentially driving a plurality of signal transmitting elements of a transmitter via a controller; there are included angles between each of the signal transmitting elements and a signal receiving element of a receiver, and the included angles are different from one another; receiving an echo signal from each of the signal transmitting elements and transmitting the echo signals to the controller by the signal receiving element; comparing the echo signals and selecting one of the signal transmitting elements as a target signal transmitting element according to the comparison result by the controller; and generating a measurement result according to the echo signal of the target signal transmitting element by the controller.

Further scope of applicability of the present application will become more apparent from the detailed description given hereinafter. However, it should be understood that the detailed description and specific examples, while indicating exemplary embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The present disclosure will become more fully understood from the detailed description given herein below and the accompanying drawings which are given by way of illustration only, and thus are not limitative of the present disclosure and wherein:

FIG. 1 is a stereogram of an ultrasound probe in accordance with a first embodiment of the present disclosure.

FIG. 2 is a side view of the ultrasound probe in accordance with the first embodiment of the present disclosure.

FIG. 3 is a schematic view of an operational principle of the ultrasound probe in accordance with the first embodiment of the present disclosure.

FIG. 4 is a first schematic view of the ultrasound probe in accordance with the first embodiment of the present disclosure.

FIG. 5 is a second schematic view of the ultrasound probe in accordance with the first embodiment of the present disclosure.

FIG. 6 is a stereogram of an ultrasound probe in accordance with a second embodiment of the present disclosure.

FIG. 7 is a side view of the ultrasound probe in accordance with the second embodiment of the present disclosure.

FIG. 8 is a block diagram of the ultrasound probe in accordance with a third embodiment of the present disclosure.

FIG. 9 is a flow chart of a control method of the ultrasound probe in accordance with the third embodiment of the present disclosure.

FIG. 10 is a flow chart of a control logic mechanism of the ultrasound probe in accordance with the third embodiment of the present disclosure.

FIG. 11A-FIG. 11C are measurement results of the ultrasound probe in accordance with the third embodiment of the present disclosure.

### DETAILED DESCRIPTION

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

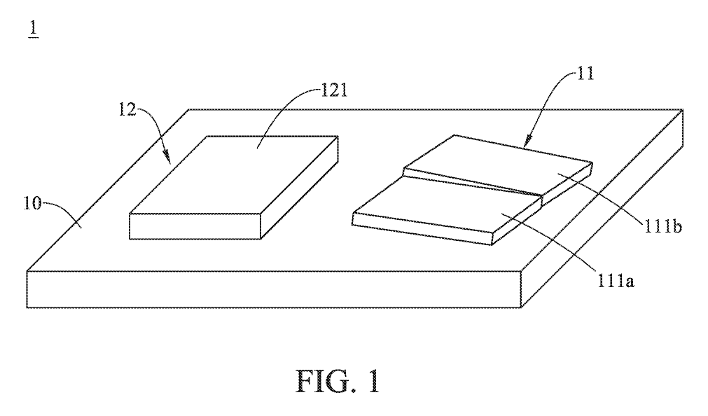

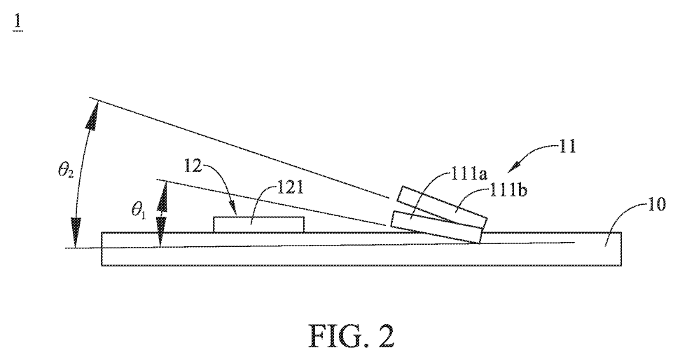

Please refer to FIG. 1 and FIG. 2, which are a stereogram and a side view of an ultrasound probe in accordance with a first embodiment of the present disclosure respectively. As shown in FIG. 1, the ultrasound probe 1 includes a substrate 10, a transmitter 11 and a receiver 12.

The transmitter 11 is disposed on the substrate 10 and the transmitter 11 includes two signal transmitting elements 111a, 111b. In the embodiment, the signal transmitting elements 111a, 111b may be sound transmitting elements, such as piezoelectric plates. The signals emitted by the sound transmitting elements are radial acoustic waves, which are suitable for biomedical signal measurement. The frequency range of the signals emitted by the sound transmitting elements is 1˜3 MHz. More specifically, the signals of low frequency have high penetration; on the contrary, the signals of high frequency have low penetration. Thus, for the biomedical measurement (e.g. measuring the heart sound of a baby or the depth of the blood vessel of a patient), the frequency of the signals emitted by the sound transmitting elements may be 2 MHz.

In another embodiment, the signal transmitting elements 111a, 111b may be light transmitting elements, such as LED light emitters, laser emitters, etc. The signals emitted by the light emitting elements are straight light beams (e.g. green light), which are suitable for other (non-biomedical) signal measurement.

The receiver 12 is disposed on the substrate 10 and the receiver 12 includes a signal receiving element 121. In the embodiment, the signal receiving element 121 is a sound sensing element, such as a piezoelectric plate, which is suitable for biomedical signal measurement.

Similarly, in another embodiment, the signal receiving element 121 may be light sensing elements, such as LED light receivers, laser receivers, etc., which are suitable for other (non-biomedical) signal measurement.

As shown in FIG. 2, there is an included angle θ₁ between the signal transmitting element 111a and the signal receiving element 121, and an included angle θ₂ between the signal transmitting element 111b and the signal receiving element 121.

In general, the included angle θ₁ and the included angle θ₂ are about 1°˜20°, and the difference between the included angle θ₁ and the included angle θ₂ is about 3°˜4°. For example, in the embodiment, the included angle θ₁ may be 5° and the included angle θ₂ may be 8°. In another embodiment, the included angle θ₁ may be 6° and the included angle θ₂ may be 10°. The included angle θ₁ and the included angle θ₂ can be designed according to the characteristics of the target object G in order to obtain the best measurement result.

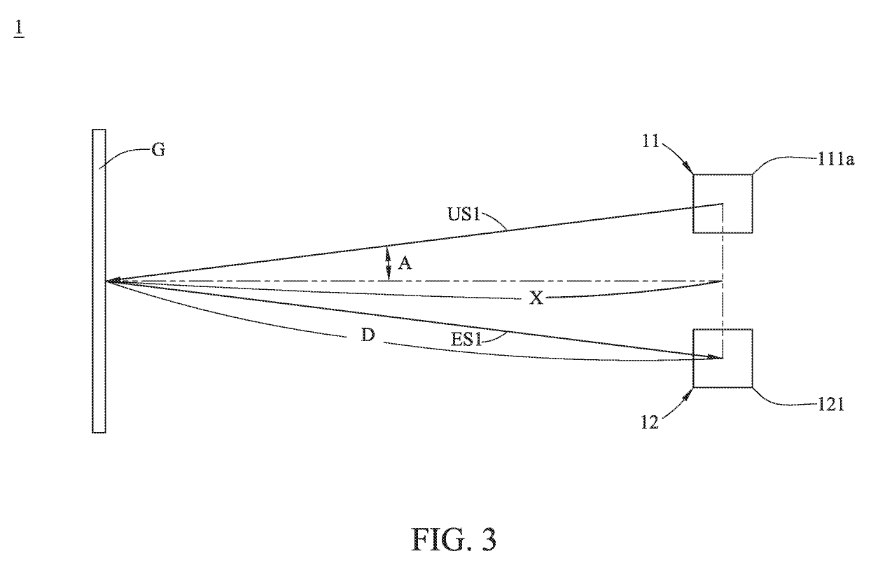

Please refer to FIG. 3, which is a schematic view of an operational principle of the ultrasound probe in accordance with the first embodiment of the present disclosure. FIG. 3 illustrates the operational principle of the ultrasound probe 1 by taking the signal transmitting element 111a as an example.

The signal transmitting element 111a transmits an ultrasound signal US1 to a target object G, and the ultrasound signal US1 is reflected by the target object G to generate an echo signal ES1.

The signal receiving element 121 receives the echo signal ES1.

The propagation velocity of sound in the air is related to temperature, as shown in Equation (1), as follows:

v=331+0.6T (1)

In Equation (1), v stands for the propagation velocity (m/s) of sound in the air; T stands for temperature (° C.).

The path length that the echo signal ES1 is transmitted from the target object G to the signal receiving element 121 is as shown in Equation (2), as follows:

D=v(Δt/2) (2)

In Equation (2), D stands for the path length (m) that the echo signal ES1 is transmitted from the target object G to the signal receiving element 121; Δt stands for the time (s) difference from a time of the signal transmitting element 111 transmitting the ultrasound signal US1 to a time of the signal receiving element 121 receiving the echo signal ES1.

Thus, the distance between the ultrasound probe 1 and the target object G is as shown in Equation (3), as follows:

X=D cos A=v(Δt/2)cos A (3)

In Equation (3), X stands for the distance (m) between the ultrasound probe 1 and the target object G; A stands for the incident angle of the ultrasound signal US1.

Therefore, via the above mechanism, the ultrasound probe 1 can calculate the distance X between the ultrasound probe 1 and the target object G according to the echo signal ES1.

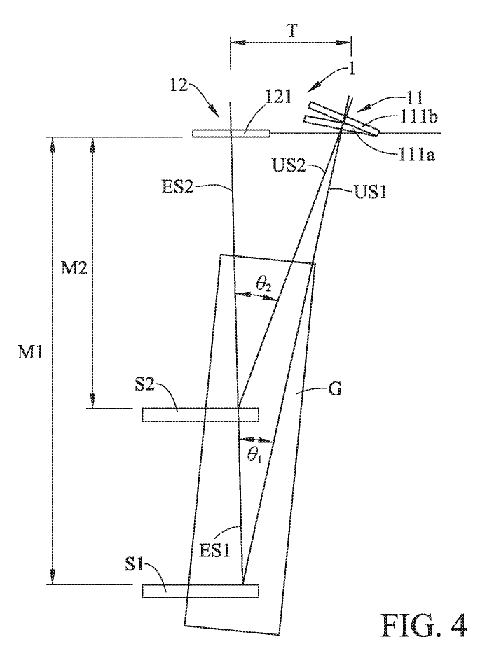

Please refer to FIG. 4, which is a first schematic view of the ultrasound probe in accordance with the first embodiment of the present disclosure. As shown in FIG. 4, since the included angles of the signal transmitting elements 111a, 111b are different from each other, so the ultrasound probe 1 can perform the measurement for the target objects in different distances or depths.

The signal transmitting element 111a transmits an ultrasound signal US1 to the target object G, and the ultrasound signal US1 is reflected by the reflection surface Si to generate an echo signal ES1. The echo signal ES1 is transmitted to the signal receiving element 121.

The signal transmitting element 111b transmits an ultrasound signal US2 to the target object G, and the ultrasound signal US2 is reflected by the reflection surface S2 to generate an echo signal ES2. The echo signal ES2 is transmitted to the signal receiving element 121.

The distance between the ultrasound probe 1 and the reflection surface S1 is M1, which can be calculated according to the included angle θ₁. The included angle θ₁ is between the signal transmitting element 111a and the signal receiving element 121 Similarly, the distance between the ultrasound probe 1 and the reflection surface S2 is M2, which can be calculated according to the included angle θ₂. The included angle θ₂ is between the signal transmitting element 111b and the signal receiving element 121. More specifically, the distance M1 between the ultrasound probe 1 and the reflection surface S1 can be calculated according to Equation (4), as follows:

tan θ₁=T/M1 (4)

In Equation (4), T stands for the distance between the center of the signal transmitting element 111a and the center of the signal receiving element 121. For example, if θ₁ is 5° and T is 10.5 mm, M1 is 120 mm according to the calculation result of Equation (4). If θ₁ is 5° and T is 7.5 mm, M1 is 86 mm according to the calculation result of Equation (4).

Similarly, the distance M2 between the ultrasound probe 1 and the reflection surface S2 can be calculated according to Equation (5), as follows:

Tan θ₂=T/M2 (5)

In Equation (5), T stands for the distance between the center of the signal transmitting element 111b and the center of the signal receiving element 121 (in the embodiment, the distance between the center of the signal transmitting element 111a and the center of the signal receiving element 121 is equal to the distance between the center of the signal transmitting element 111b and the center of the signal receiving element 121.). For example, if θ₂ is 8° and T is 10.5 mm, M2 is 75 mm according to the calculation result of Equation (5). If θ₂ is 8° and T is 7.5 mm, M2 is 53 mm according to the calculation result of Equation (5).

Therefore, via the special structure design of the ultrasound probe 1, the ultrasound probe 1 can effectively perform the measurement for the target objects in different distances or depths, so the detection blind zone can be effectively reduced. In this way, the performance of the ultrasound probe 1 can be significantly enhanced, so the ultrasound probe 1 is very suitable for parking sensors or other general applications. In addition, the ultrasound probe 1 can further effectively measure blood flow velocity according to Doppler Effect.

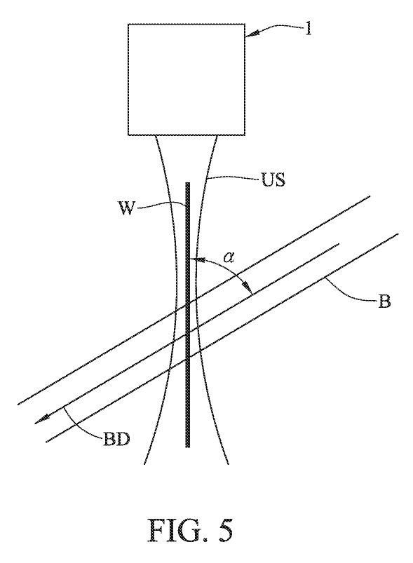

Please refer to FIG. 5, which is a second schematic view of the ultrasound probe in accordance with the first embodiment of the present disclosure. As shown in FIG. 5, the ultrasound probe 1 transmits an ultrasound signal US to a blood vessel B, and the ultrasound probe 1 receives an echo signal to measure the blood flow velocity. More specifically, according to Doppler Effect, the blood flow velocity is as shown in Equation (6), as follows:

V_{b}=(F_{D}×C)/(2F_{O}×Cos α) (6)

In Equation (6), V_{b} stands for the blood flow velocity; F_{D} stands for the Doppler shift; C stands for the velocity of the sound in the tissue; F_{O} stands for the original frequency of the ultrasound signal US; a stands for the included angle between the beam W of the ultrasound signal US and the blood flow direction BD. Since the Doppler shift occurs twice, the original frequency F_{O} of the ultrasound signal US should be multiplied by 2. Besides, Cosa is used to compensate for the included angle a between the beam W of the ultrasound signal US and the blood flow direction BD.

Via Equation (6), the ultrasound probe 1 can effectively measure the blood flow velocity V_{b} according to Doppler Effect.

As described above, the ultrasound probe 1 of the embodiment has a special structure design. If the target object G in FIG. 4 is a blood vessel, the ultrasound probe 1 can also effectively measure the blood flow velocity of the blood vessel in different depths. Thus, the ultrasound probe 1 can measure the blood flow velocity of different patients at high accuracy even if the blood vessels of these patients are in different depths. And the ultrasound probe 1 provides various data according to the blood flow velocities. These data include, for example, stroke volume, stroke volume index, stroke volume variability, stroke work, cardiac output, cardiac index, cardiac power, blood pressure, heart rate, peak velocity flow, velocity time integral, minute distance, ejection time percent, systemic vascular resistance, systemic vascular resistance index, mean pressure gradient, flow time, flow time corrected, etc. Moreover, the ultrasound probe 1 is also applicable to other medical applications.

The embodiment just exemplifies the present disclosure and is not intended to limit the scope of the present disclosure. Any equivalent modification and variation according to the spirit of the present disclosure is to be also included within the scope of the following claims and their equivalents.

It is worthy to point out that currently available ultrasound probes can only detect a target object in a fixed distance due to the limits in structure thereof. Such that detection blind zones are prone to occur when the distance between the ultrasound probes and the target object changes, for instance, the distance is too close or too deep. Thus, the currently available ultrasound probes cannot effectively measure the distance between them and the target object. On the contrary, according to one embodiment of the present disclosure, the transmitter 11 of the ultrasound probe 1 includes a plurality of signal transmitting elements 111a, 111b. Besides, the included angles θ₁, θ₂ between the signal transmitting elements 111a, 111b and the signal receiving element 121 of the receiver 12 are different from each other. Therefore, the ultrasound probe 1 can effectively detect the target objects G in different distances or depths, and the ultrasound probe 1 can precisely detect the target objects G close thereto, so the detection blind zones can be reduced.

Moreover, since different patients have different body types and their blood vessel depths may be different, the currently available ultrasound probes cannot correctly measure the blood flow velocities of different patients when being applied to blood flow velocity measurement or other medical applications. On the contrary, according to one embodiment of the present disclosure, the ultrasound probe 1 has a special structure design, so the target objects G in different distances or depths can be effectively detected. Accordingly, the ultrasound probe 1 can precisely measure the blood flow velocities of different patients even if the blood vessels of these patients are in different depths. Therefore, the ultrasound probe 1 is very suitable for blood flow velocity measurement or other medical applications.

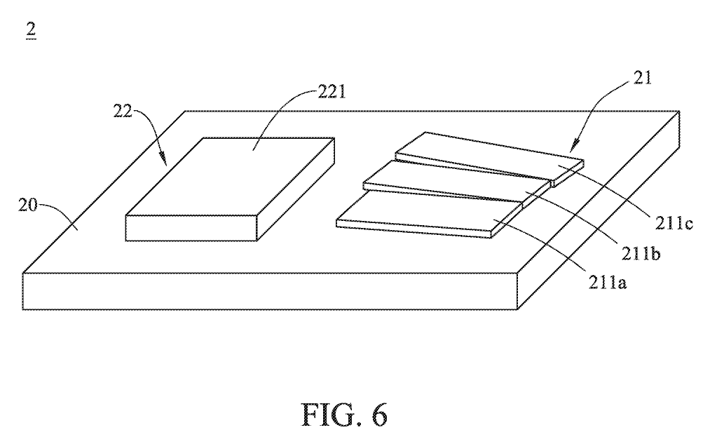

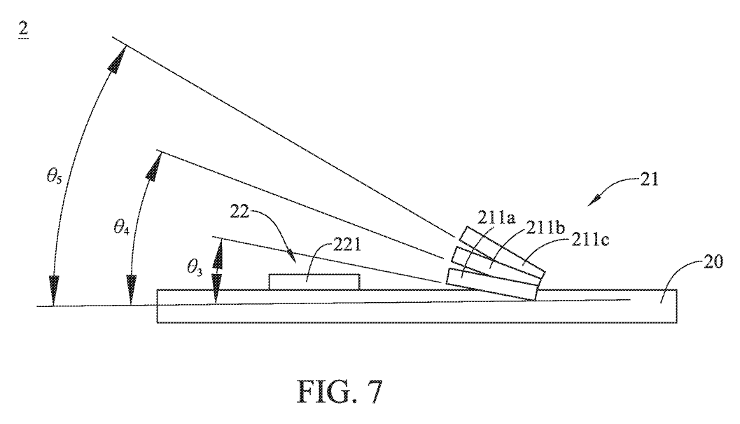

Please refer to FIG. 6 and FIG. 7, which are a stereogram and a side view of an ultrasound probe in accordance with a second embodiment of the present disclosure respectively. As shown in FIG. 6, the ultrasound probe 2 includes a substrate 20, a transmitter 21 and a receiver 22.

The receiver 22 is disposed on the substrate 20, and the receiver 22 includes a signal receiving element 221.

The transmitter 21 is disposed on the substrate 20. The difference between this embodiment and the previous embodiment is that the transmitter 21 includes three signal transmitting elements 211a, 211b, 211c.

As shown in FIG. 7, there is an included angle θ₃ between the signal transmitting element 211a and the signal receiving element 221, an included angle θ₄ between the signal transmitting element 211b and the signal receiving element 221, and an included angle θ₅ between the signal transmitting element 211c and the signal receiving element 221.

The included angle θ₄ between the signal transmitting element 211b and the signal receiving element 221 is greater than the included angle θ₃ between the signal transmitting element 211a and the signal receiving element 221. The included angle θ₅ between the signal transmitting element 211c and the signal receiving element 221 is greater than the included angle θ₄ between the signal transmitting element 211b and the signal receiving element 221.

Similarly, the included angle θ₃, the included angle θ₄ and the included angle θ₅ are, in general, about 1°˜20°. The difference between the included angle θ₄ and the included angle θ₃, and the difference between the included angle θ₅ and the included angle θ₄ are about 3°˜4°. For example, in the embodiment, the included angle θ₃ may be 3°; the included angle θ₄ may be 6°; the included angle θ₅ may be 9°. In another embodiment, the included angle θ₃ may be 5°; the included angle θ₄ may be 9°; the included angle θ₅ may be 13°. The included angle θ₃, the included angle θ₄ and the included angle θ₅ can be designed according to the characteristics of the target object G in order to obtain the best measurement result.

As described above, the ultrasound probe 2 can have more signal transmitting elements to effectively detect more target objects G in different distances or depths so as to satisfy the actual requirements.

The embodiment just exemplifies the present disclosure and is not intended to limit the scope of the present disclosure. Any equivalent modification and variation according to the spirit of the present disclosure is to be also included within the scope of the following claims and their equivalents.

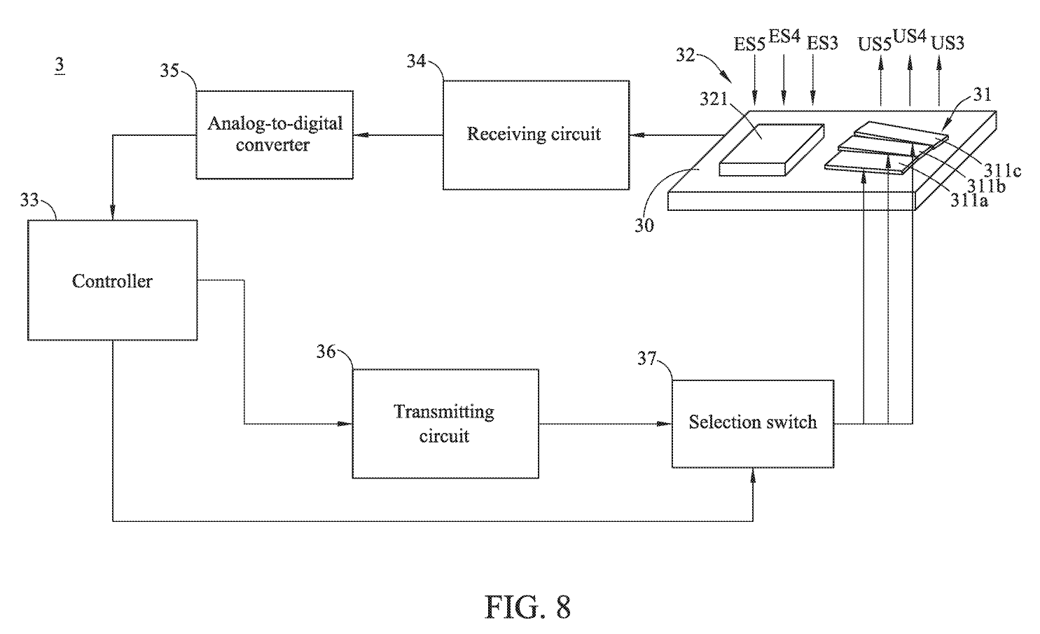

Please refer to FIG. 8, which are a block diagram of the ultrasound probe in accordance with a third embodiment of the present disclosure. As shown in FIG. 8, similarly, the ultrasound probe 3 includes a substrate 30, a transmitter 31 and a receiver 32.

The receiver 32 is disposed on the substrate 30, and the receiver 32 includes a signal receiving element 321. The transmitter 31 is disposed on the substrate 30, and the transmitter 31 includes a plurality of signal transmitting elements 311a, 311b, 311c. The structure of the ultrasound probe 3 is similar to the previous embodiment, so will not be described herein.

The difference between this embodiment and the previous embodiment is that the ultrasound probe 3 further includes a controller 33, a receiving circuit 34, an analog-to-digital converter 35, a transmitting circuit 36 and a selection switch 37.

The controller 33 selects the signal transmitting element 311a via the selection switch 37, and the controller 33 drives the signal transmitting element 311a to emit an ultrasound signal US3 via the transmitting circuit 36. The receiving circuit 34 receives the echo signal ES3 of the ultrasound signal US3 via the signal receiving element 321. And the analog-to-digital converter 35 converts the echo signal ES3 into a digital signal. The controller 33 receives and saves the digital signal.

Similarly, the controller 33 selects the signal transmitting element 311b via the selection switch 37, and the controller 33 drives the signal transmitting element 311b to emit an ultrasound signal US4 via the transmitting circuit 36. The receiving circuit 34 receives the echo signal ES4 of the ultrasound signal US4 via the signal receiving element 321. And the analog-to-digital converter 35 converts the echo signal ES4 into a digital signal. The controller 33 receives and saves the digital signal.

Next, the controller 33 selects the signal transmitting element 311c via the selection switch 37, and the controller 33 drives the signal transmitting element 311c to transmit an ultrasound signal US5 via the transmitting circuit 36. The receiving circuit 34 receives the echo signal ES5 of the ultrasound signal US5 via the signal receiving element 321. And the analog-to-digital converter 35 converts the echo signal ES5 into a digital signal. The controller 33 receives and saves the digital signal.

Afterwards, the controller 33 compares the signal characteristics of the echo signals, ES3, ES4, ES5. In the embodiment, the above signal characteristics are signal strengths. The controller 33 compares the signal strengths of the echo signals, ES3, ES4, ES5, and the controller 33 selects the signal transmitting element having the echo signal with the highest signal strength as the target signal transmitting element. Then, the controller 33 generates a measurement result according to the echo signal of the target signal transmitting element. In another embodiment, the above signal characteristics may be signal waveforms or other relevant characteristics. For example, if the comparison result shows that the echo signal ES3 of the signal transmitting element 311a has the highest signal strength, the controller 33 selects the signal transmitting element 311a as the target signal transmitting element, and the controller 33 generates the measurement result according to the echo signal ES3 thereof.

The controller 33 keeps switching the signal transmitting elements 311a, 311b, 311c via the selection switch 37 in order to scan the target object G. And the controller 33 re-selects the target signal transmitting element having the echo signal with the highest signal strength from the signal transmitting elements 311a, 311b, 311c. In this way, the precision of the measurement result can be further increased.

Via the above special logic control mechanism, the ultrasound probe 3 can accurately select the most proper target signal transmitting element from the signal transmitting elements 311a, 311b, 311c. Besides, the ultrasound probe 3 can keep scanning so as to re-select the target signal transmitting element with the highest signal strength of the echo signal among the signal transmitting elements 311a, 311b, 311c so as to optimize the measurement result. Therefore, when being applied to blood flow velocity measurement, the ultrasound probe 3 can measure the blood flow velocities of different patients at high accuracy even if the blood vessels of these patients are in different depths.

The embodiment just exemplifies the present disclosure and is not intended to limit the scope of the present disclosure. Any equivalent modification and variation according to the spirit of the present disclosure is to be also included within the scope of the following claims and their equivalents.

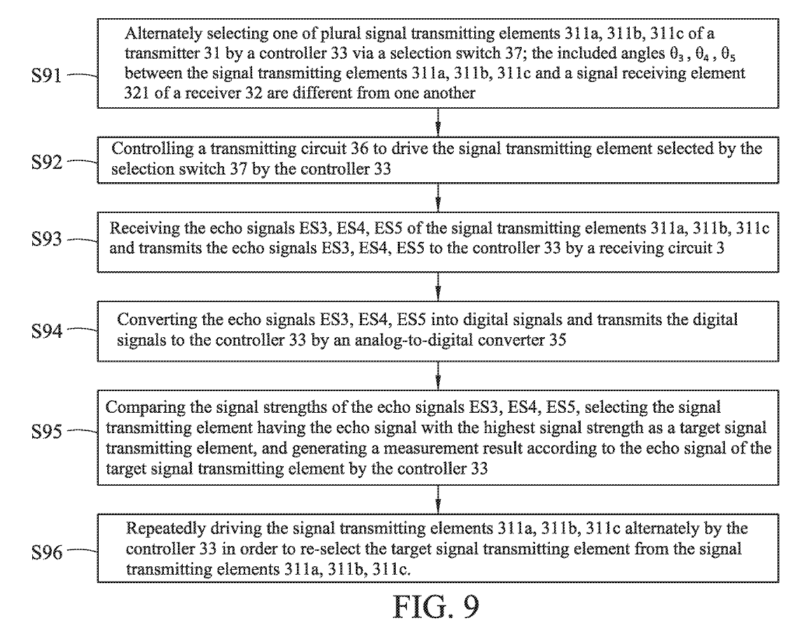

Please refer to FIG. 9, which is a flow chart of a control method of the ultrasound probe in accordance with the third embodiment of the present disclosure. The control method of the ultrasound probe 3 of the embodiment includes the following steps:

Step S91: a controller 33 alternately selects one of plural signal transmitting elements 311a, 311b, 311c of a transmitter 31 via a selection switch 37. The included angles θ₃, θ₄, θ₅ between the signal transmitting elements 311a, 311b, 311c and a signal receiving element 321 of a receiver 32 are different from one another.

Step S92: the controller 33 controls a transmitting circuit 36 to drive the signal transmitting element selected by the selection switch 37.

Step S93: a receiving circuit 34 receives the echo signals ES3, ES4, ES5 of the signal transmitting elements 311a, 311b, 311c and transmits the echo signals ES3, ES4, ES5 to the controller 33.

Step S94: an analog-to-digital converter 35 converts the echo signals ES3, ES4, ES5 into digital signals and transmits the digital signals to the controller 33.

Step S95: the controller 33 compares the signal strengths of the echo signals ES3, ES4, ES5, selects the signal transmitting element having the echo signal with the highest signal strength as a target signal transmitting element. And the controller 33 generates a measurement result according to the echo signal of the target signal transmitting element.

Step S96: the controller 33 repeatedly drives the signal transmitting elements 311a, 311b, 311c alternately in order to re-select the target signal transmitting element from the signal transmitting elements 311a, 311b, 311c.

More specifically, the controller 33 selects the signal transmitting element 311a first, and the controller 33 controls the transmitting circuit 36 to drive the signal transmitting element 311a. After the receiving circuit 34 receives the echo signal ES3 of the signal transmitting element 311a, the analog-to-digital converter 35 converts the echo signal ES3 into a digital signal, and the analog-to-digital converter 35 transmits the digital signal to the controller 33. Then, the controller 33 selects the signal transmitting element 311b, and the controller 33 controls the transmitting circuit 36 to drive the signal transmitting element 311b. After the receiving circuit 34 receives the echo signal ES4 of the signal transmitting element 311b, the analog-to-digital converter 35 converts the echo signal ES4 into a digital signal and transmits the digital signal to the controller 33. Afterwards, the controller 33 selects the signal transmitting element 311c, and the controller 33 controls the transmitting circuit 36 to drive the signal transmitting element 311c. After the receiving circuit 34 receives the echo signal ES5 of the signal transmitting element 311c, the analog-to-digital converter 35 converts the echo signal ES5 into a digital signal, and the analog-to-digital converter 35 transmits the digital signal to the controller 33. Finally, the controller 33 performs the comparison process to generate the measurement result. And the controller 33 keeps alternately driving the signal transmitting elements 311a, 311b, 311c via the above mechanism in order to re-select the target signal transmitting element with the highest signal strength of the echo signal from the signal transmitting elements 311a, 311b, 311c.

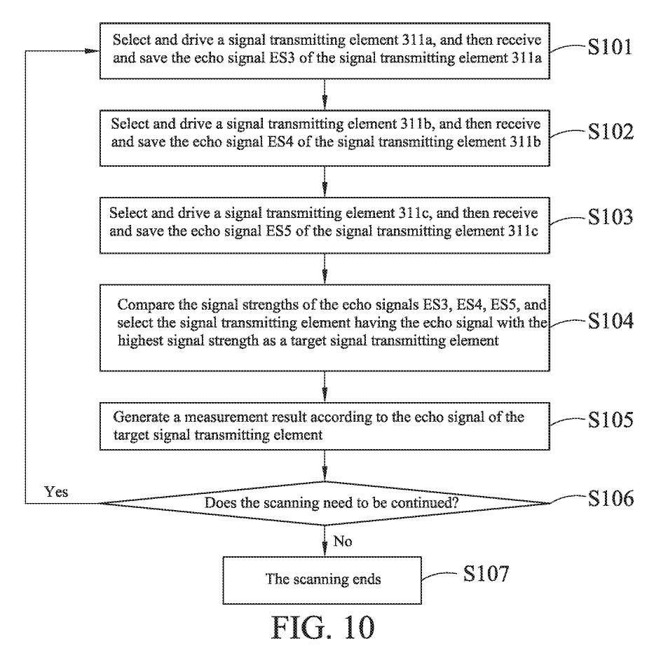

Please refer to FIG. 10, which is a flow chart of a control logic mechanism of the ultrasound probe in accordance with the third embodiment of the present disclosure. FIG. 10 describes the detailed process of the control logic mechanism of the ultrasound probe 3 of the embodiment, which includes the following steps:

Step S101: select and drive a signal transmitting element 311a, and then receive and save the echo signal ES3 of the signal transmitting element 311a; then, the process proceeds to Step S102.

Step S102: select and drive a signal transmitting element 311b, and then receive and save the echo signal ES4 of the signal transmitting element 311b; then, the process proceeds to Step S103.

Step S103: select and drive a signal transmitting element 311c, and then receive and save the echo signal ES5 of the signal transmitting element 311c; then, the process proceeds to Step S104.

Step S104: compare the signal strengths of the echo signals ES3, ES4, ES5, and select the signal transmitting element having the echo signal with the highest signal strength as a target signal transmitting element; then, the process proceeds to Step S105.

Step S105: generate a measurement result according to the echo signal of the target signal transmitting element; then, the process proceeds to Step S106.

Step S106: does the scanning need to be continued? if it does, the process returns to Step S101; if it does not, the process proceeds to Step S107.

Step S107: the scanning ends.

The embodiment just exemplifies the present disclosure and is not intended to limit the scope of the present disclosure. Any equivalent modification and variation according to the spirit of the present disclosure is to be also included within the scope of the following claims and their equivalents.

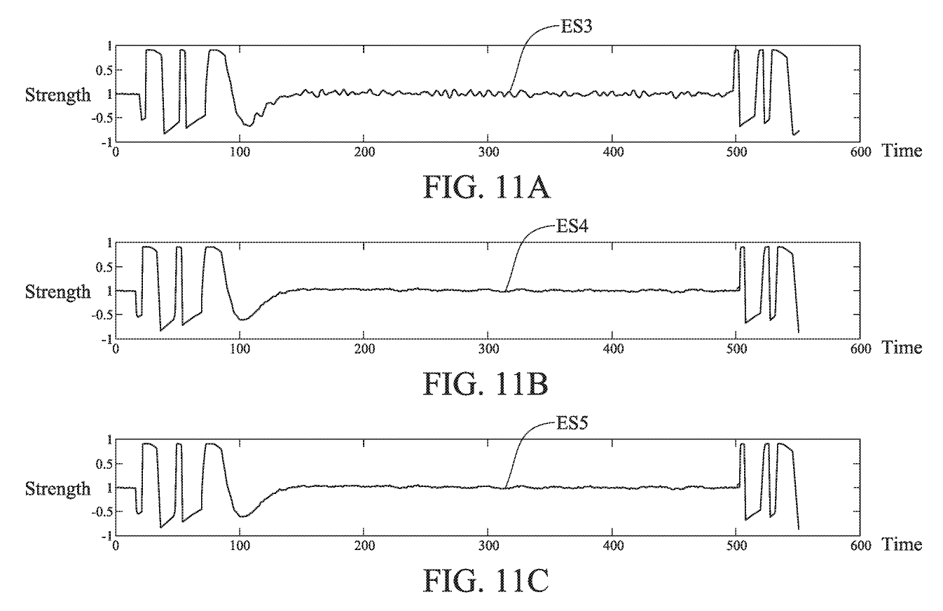

Please refer to FIG. 11A, FIG. 11B and FIG. 11C, which are measurement results of the ultrasound probe 3 measured from the target objects G in accordance with the third embodiment of the present disclosure.

First, the controller 33 sequentially selects, and the controller 33 drives the signal transmitting elements 311a, 311b, 311c. Then, the receiving circuit 34 respectively receives the echo signal ES3 of the signal transmitting element 311a, the echo signal ES4 of the signal transmitting element 311b and the echo signal ES5 of the signal transmitting element 311c.

FIG. 11A shows the waveform of the echo signal ES3. As shown in FIG. 11A, the horizontal axis is time (second) and the vertical axis is intensity; the intensity is voltage value (volt) after normalization. The waveform in the middle portion of the echo signal ES3 shows that the echo signal ES3 obviously changes, which can be considered that the echo signal ES3 has higher strength (compared with the echo signals ES4, ES5). Thus, the signal transmitting element 311a can effectively measure the target object G.

FIG. 11B shows the waveform of the echo signal ES4. As shown in FIG. 11B, the horizontal axis is time and the vertical axis is intensity. The waveform in the middle portion of the echo signal ES4 shows that intensity of the echo signal ES4 is 0. Therefore, the signal transmitting element 311b cannot effectively measure the target object G.

FIG. 11C shows the waveform of the echo signal ES5. As shown in FIG. 11C, the horizontal axis is time and the vertical axis is intensity. The waveform in the middle portion of the echo signal ES5 shows that intensity of the echo signal ES5 is 0. Therefore, the signal transmitting element 311c cannot effectively detect the target object G.

Since the signal transmitting element 311a can effectively detect the target object G, the controller 33 selects the signal transmitting element 311a as the target signal transmitting element. The controller 33 keeps repeating the above process in order to re-select the target signal transmitting element from the signal transmitting elements 311a, 311b, 311c according to the measurement results of the signal transmitting elements 311a, 311b, 311c.

The embodiment just exemplifies the present disclosure and is not intended to limit the scope of the present disclosure. Any equivalent modification and variation according to the spirit of the present disclosure is to be also included within the scope of the following claims and their equivalents.

In summation of the description above, according to one embodiment of the present disclosure, the transmitter 31 of the ultrasound probe 3 includes plural signal transmitting elements 311a, 311b, 311c. Besides, the included angles θ₃, θ₄, θ₅ between the signal transmitting elements 311a, 311b, 311c and the signal receiving element 321 of the receiver 32 are different from one another. Therefore, the ultrasound probe 3 can effectively detect the target objects G in different distances or depths, and can precisely detect the target objects G close thereto, so the detection blind zones can be reduced.

Moreover, according to one embodiment of the present disclosure, the ultrasound probe 3 has a special structure design, so the target objects G in different distances or depths can be effectively detected. Accordingly, the ultrasound probe 3 can precisely measure the blood flow velocities of different patients even if the blood vessels of these patients are in different depths. Therefore, the ultrasound probe 3 is very suitable for blood flow velocity measurement or other medical applications.

Furthermore, according to one embodiment of the present disclosure, the ultrasound probe 3 has a special logic control mechanism. Such that the ultrasound probe 3 can accurately select the most proper target signal transmitting element from the signal transmitting elements 311a, 311b, 311c, which can significantly increase the precision of the measurement result.

It will be apparent to those skilled in the art that various modifications and variations can be made to the disclosed embodiments. It is intended that the specification and examples be considered as exemplary only, with a true scope of the disclosure being indicated by the following claims and their equivalents.

What is claimed is:

1. An ultrasound probe, comprising:
a receiver, comprising a signal receiving element; and
a transmitter, comprising a plurality of signal transmitting elements, wherein there are an included angles between each of the signal transmitting elements and the signal receiving element, and the included angles are different from one another; and
a controller, connected to the transmitter and the receiver, and configured to sequentially drives the signal transmitting elements, and the signal receiving element configured to receive an echo signal from each of the signal transmitting elements;
wherein the controller makes a comparison among the echo signals and selects one of the signal transmitting elements as a target signal transmitting element according to the comparison result, whereby the controller generates a measurement result according to the echo signal of the target signal transmitting element.

2. The ultrasound probe of claim 1, wherein the controller compares signal characteristics of the echo signals and selects one of the signal transmitting elements as the target signal transmitting element according to the signal characteristics of the echo signals.

3. The ultrasound probe of claim 1, wherein the controller compares signal strengths of the echo signals and selects the signal transmitting element having a highest signal strength of the echo signal as the target signal transmitting element.

4. The ultrasound probe of claim 1, wherein the controller compares signal waveforms of the echo signals and selects one of the signal transmitting elements as the target signal transmitting element according to the signal waveforms of the echo signals.

5. The ultrasound probe of claim 1, wherein the controller repeatedly and alternatively drives the signal transmitting elements in order to re-select the target signal transmitting element from the signal transmitting elements.

6. The ultrasound probe of claim 1, further comprising:
a receiving circuit, connected to the transmitter and configured to receive the echo signals; and
an analog-to-digital converter, connected to the receiving circuit and the controller, and the analog-to-digital converter configured to receive the echo signals, convert the echo signals into digital signals, and transmit the digital signals to the controller.

7. The ultrasound probe of claim 1, further comprising a selection switch, wherein the controller is connected to the transmitter via the selection switch and alternately selects the signal transmitting elements via the selection switch.

8. The ultrasound probe of claim 7, further comprising a transmitting circuit connected to the controller and the selection switch, wherein the controller controls the transmitting circuit to drive the signal transmitting element selected by the selection switch.

9. The ultrasound probe of claim 1, wherein the signal transmitting elements and the signal receiving elements are piezoelectric plates.

10. The ultrasound probe of claim 1, wherein the measurement result is a blood flow velocity.

11. A control method for an ultrasound probe, comprising:
sequentially driving a plurality of signal transmitting elements of a transmitter via a controller, wherein there are included angles between each of the signal transmitting elements and a signal receiving element of a receiver, and the included angles are different from one another;
receiving an echo signal from each of the signal transmitting elements and transmitting the echo signals to the controller by the signal receiving element;
comparing the echo signals and selecting one of the signal transmitting elements as a target signal transmitting element according to a comparison result by the controller; and
generating a measurement result according to the echo signal of the target signal transmitting element by the controller.

12. The control method of claim 11, wherein the step of comparing the echo signals and selecting one of the signal transmitting elements as the target signal transmitting element according to the comparison result by the controller further comprising:
comparing signal characteristics of the echo signals and selecting one of the signal transmitting elements as the target signal transmitting element according to the signal characteristics of the echo signals by the controller.

13. The control method of claim 11, wherein the step of comparing the echo signals and selecting one of the signal transmitting elements as the target signal transmitting element according to the comparison result by the controller further comprising:
comparing signal strengths of the echo signals and selecting the signal transmitting element having a highest signal strength of the echo signal as the target signal transmitting element by the controller.

14. The control method of claim 11, wherein the step of comparing the echo signals and selecting one of the signal transmitting elements as the target signal transmitting element according to the comparison result by the controller further comprising:
comparing signal waveforms of the echo signals and selecting one of the signal transmitting element as the target signal transmitting element according to the signal waveforms of the echo signals by the controller.

15. The control method of claim 11, further comprising:
repeatedly and alternatively driving the signal transmitting elements to re-select the target signal transmitting element from the signal transmitting elements by the controller.

16. The control method of claim 11, wherein the step of receiving the echo signal from each of the signal transmitting elements and transmitting the echo signals to the controller by the signal receiving element further comprising:
receiving the echo signals by a receiving circuit; and
converting the echo signals into digital signals and transmitting the digital signals to the controller by an analog-to-digital converter.

17. The control method of claim 11, wherein the step of sequentially driving the plurality of signal transmitting elements of the transmitter via the controller further comprising:
alternately selecting the signal transmitting elements of the transmitter by the controller via a selection switch; and
controlling a transmitting circuit to drive the signal transmitting element selected by the selection switch by the controller.
